(19) United States
(12) Patent Application Publication
(10) Pub. No.: US 2011/0144140 A1
(43) Pub. Date: Jun. 16, 2011

(54) PYRIDINYL-PYRIMIDINE DERIVATIVES USEFUL AS POTASSIUM CHANNEL MODULATING AGENTS

(76) Inventors: Birgitte L. Eriksen, Farum (DK); Ulrik Svane Sorensen, Seborg (DK); Charlotte Hougaard, Bagsvaerd (DK); Lene Teuber, Vaerlose (DK); Dan Peters, Malmo (SE); Falle Christophersen, Ballerup (DK); Tina Holm Johansen, Smorum (DK)

(21) Appl. No.: 12/377,392

(22) PCT Filed: Sep. 6, 2007
(86) PCT No.: PCT/EP07/59313

§ 371 Date: Feb. 13, 2009

Related U.S. Application Data
(60) Provisional application No. 60/824,816, filed on Sep. 7, 2006.

(30) Foreign Application Priority Data
Sep. 7, 2006 (DK) PA 2006 01151

(51) Int. Cl. A61K 31/506; C07D 401/04; A61P 11/00; A61P 13/00; A61P 15/10; A61P 25/22; A61P 25/08; A61P 25/18; A61P 29/00

(52) U.S. Cl. 514/269; 544/319; 544/333; 514/256; 544/328

(57) ABSTRACT

This invention relates to novel pyridinyl-pyrimidine derivatives and their use as potassium channel modulating agents. Moreover the invention is directed to pharmaceutical compositions useful for the treatment or alleviation of diseases or disorders associated with the activity of potassium channels.

### TECHNICAL FIELD

This invention relates to novel pyridinyl-pyrimidine derivatives and their use as potassium channel modulating agents. Moreover the invention is directed to pharmaceutical compositions useful for the treatment or alleviation of diseases or disorders associated with the activity of potassium channels.

### BACKGROUND ART

Ion channels are transmembrane proteins, which catalyse the transport of inorganic ions across cell membranes. The ion channels participate in processes as diverse as the generation and timing of action potentials, synaptic transmissions, secretion of hormones, contraction of muscles, etc.

All mammalian cells express potassium (K⁺) channels in their cell membranes, and the channels play a dominant role in the regulation of the membrane potential. In nerve and muscle cells they regulate the frequency and form of the action potential, the release of neurotransmitters, and the degree of broncho- and vasodilation.

From a molecular point of view, the K⁺ channels represent the largest and most diverse group of ion channels. For an overview they can be divided into five large subfamilies: Voltage-activated K⁺ channels (KO, long QT related K⁺ channels (KvLQT), inward rectifiers (K_{IR}), two-pore K⁺ channels (K_{TP}), and calcium-activated K⁺ channels (K_{ca}).

The latter group, the Ca²⁺-activated K⁺ channels, consists of three well-defined subtypes: SK channels, IK channels and BK channels. SK, IK and BK refer to the single-channel conductance (Small, Intermediate and Big conductance K channel). The SK, IK, and BK channels exhibit differences in e.g. voltage- and calcium-sensitivity, pharmacology, distribution and function.

SK channels are present in many central neurons and ganglia, where their primary function is to hyperpolarize nerve cells following one or several action potentials, in order to prevent long trains of epileptogenic activity to occur. The SK channels are also present in several peripheral cells including skeletal muscle, gland cells, liver cells, and T-lymphocytes. The significance of SK channels in normal skeletal muscle is not clear, but their number is significantly increased in denervated muscle, and the large number of SK channels in the muscle of patients with myotonic muscle dystrophia, suggest a role in the pathogenesis of the disease.

Studies indicate that K⁺ channels may be a therapeutic target in the treatment of a number of diseases including asthma, cystic fibrosis, chronic obstructive pulmonary disease and rhinorrhea, convulsions, vascular spasms, coronary artery spasms, renal disorders, polycystic kidney disease, bladder spasms, urinary incontinence, bladder outflow obstruction, irritable bowel syndrome, gastrointestinal dysfunction, secretory diarrhoea, ischaemia, cerebral ischaemia, ischaemic heart disease, angina pectoris, coronary heart disease, traumatic brain injury, psychosis, anxiety, depression, dementia, memory and attention deficits, Alzheimer's disease, dysmenorrhea, narcolepsy, Reynaud's disease, intermittent claudication, Sjorgren's syndrome, migraine, arrhythmia, hypertension, absence seizures, myotonic muscle dystrophia, xerostomi, diabetes type II, hyperinsulinemia, premature labour, baldness, cancer and immune suppression.

EP 407899 (Hoechst) discloses pyridinyl-pyrimidine derivatives useful as fungicides, and WO 2005/082884 (AstraZeneca) discloses pyridinyl-pyrimidine derivatives useful as antagonists of the metabotropic glutamate receptors (mGluR). However, use of pyridinyl-pyrimidine derivatives as modulators of SK channels and has not been reported.

### SUMMARY OF THE INVENTION

The present invention resides in the provision of novel chemical compounds capable of modulating SK channels, or subtypes of SK channels.

Accordingly, in its first aspect, the invention provides novel pyridinyl-pyrimidine derivatives of Formula I

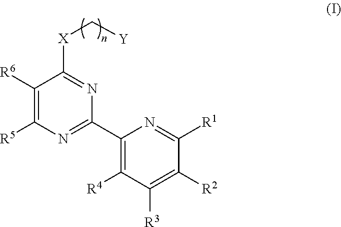

an enantiomer or a mixture of its enantiomers, an N-oxide thereof, a prodrug thereof, or a pharmaceutically acceptable salt thereof, wherein

n is 0, 1, 2 or 3;

X represents O, S or NR′; wherein

R′ represents hydrogen, alkyl, cycloalkyl or cycloalkyl-alkyl;

Y represents alkyl, cycloalkyl, alkyl-cycloalkyl, alkenyl or phenyl, which phenyl is optionally substituted one or more times with substituents selected from the group consisting of alkyl, amino-alkyl, alkyl-amino, alkyl-amino-alkyl, hydroxy-alkyl, alkoxy-alkyl, cycloalkyl, cycloalkyl-alkyl, alkenyl, halo, haloalkyl, hydroxy, alkoxy, haloalkoxy, cyano, nitro and amino; and

R¹, R², R³, R⁴, R⁵ and R⁶, independently of each other, represent hydrogen, alkyl, cycloalkyl, cycloalkyl-alkyl, alkenyl, halo, haloalkyl, hydroxy, alkoxy, haloalkoxy, alkoxy-carbonyl, cyano, nitro, amino, phenyl or benzoyl.

In another aspect, the invention provides pharmaceutical compositions comprising an effective amount of a pyridinyl-pyrimidine of the invention.

In further aspects the invention relates to the use of a pyridinyl-pyrimidine of the invention for the manufacture of a medicament for the treatment or alleviation of diseases or disorders associated with the activity of potassium channels, and to method of treatment or alleviation of disorders or conditions responsive to modulation of potassium channels.

### DETAILED DISCLOSURE OF THE INVENTION

### Potassium Channel Modulating Agents

In its first aspect, the invention provides novel pyridinyl-pyrimidine derivatives of Formula I

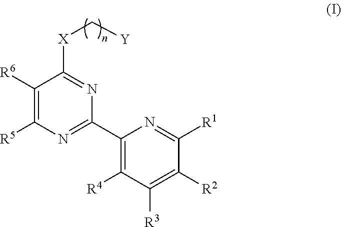

an enantiomer or a mixture of its enantiomers, an N-oxide thereof, a prodrug thereof, or a pharmaceutically acceptable salt thereof, wherein

n is 0, 1, 2 or 3;

X represents O, S or NR′; wherein R′ represents hydrogen, alkyl, cycloalkyl or cycloalkyl-alkyl;

Y represents alkyl, cycloalkyl, alkyl-cycloalkyl, alkenyl or phenyl, which phenyl is optionally substituted one or more times with substituents selected from the group consisting of alkyl, amino-alkyl, alkyl-amino, alkyl-amino-alkyl, hydroxy-alkyl, alkoxy-alkyl, cycloalkyl, cycloalkyl-alkyl, alkenyl, halo, haloalkyl, hydroxy, alkoxy, haloalkoxy, cyano, nitro and amino; and

R¹, R², R³, R⁴, R⁵ and R⁶, independently of each other, represent hydrogen, alkyl, cycloalkyl, cycloalkyl-alkyl, alkenyl, halo, haloalkyl, hydroxy, alkoxy, haloalkoxy, alkoxy-carbonyl, cyano, nitro, amino, phenyl or benzoyl.

provided, however, that the pyridinyl-pyrimidine derivative is not
4-Methyl-2-pyridin-2-yl-6-p-tolylsulfanyl-pyrimidine;
Phenyl-(2-pyridin-2-yl-6-trifluoromethyl-pyrimidin-4-yl)-amine;
(4-Chloro-phenyl)-[6-methyl-2-(6-methyl-pyridin-2-yl)-pyrimidin-4-yl]-amine; 10 or
(4-Chloro-phenyl)-(2-pyridin-2-yl-pyrimidin-4-yl)-amine.

In a preferred embodiment the pyridinyl-pyrimidine derivative of the invention is a compound of Formula I, wherein n is 0, 1, 2 or 3.

In a more preferred embodiment n is 0, 1 or 2.

In an even more preferred embodiment n is 0 or 1.

In a most preferred embodiment n is 0.

In a preferred embodiment the pyridinyl-pyrimidine derivative of the invention is a compound of Formula I, wherein X represents O, S or NR′; wherein R′ represents hydrogen, alkyl, cycloalkyl or cycloalkyl-alkyl.

In a more preferred embodiment X represents O, S or NH.

In an even more preferred embodiment X represents S or NH.

In a still more preferred embodiment X represents S.

In another still more preferred embodiment X represents NH.

In a preferred embodiment the pyridinyl-pyrimidine derivative of the invention is a compound of Formula I, wherein Y represents alkyl, cycloalkyl, alkyl-cycloalkyl, alkenyl or phenyl, which phenyl is optionally substituted one or more times with substituents selected from the group consisting of alkyl, amino-alkyl, alkyl-amino, alkyl-amino-alkyl, hydroxy-alkyl, alkoxy-alkyl, cycloalkyl, cycloalkyl-alkyl, alkenyl, halo, haloalkyl, hydroxy, alkoxy, haloalkoxy, cyano, nitro and amino.

In a more preferred embodiment Y represents cycloalkyl or phenyl, which phenyl is optionally substituted one or more times with substituents selected from the group consisting of alkyl, amino-alkyl, alkyl-amino, alkyl-amino-alkyl, hydroxy-alkyl, alkoxy-alkyl, cycloalkyl, cycloalkyl-alkyl, alkenyl, halo, haloalkyl, hydroxy, alkoxy, haloalkoxy, cyano, nitro and amino.

In an even more preferred embodiment Y represents phenyl, optionally substituted one or more times with substituents selected from the group consisting of alkyl, amino-alkyl, alkyl-amino, alkyl-amino-alkyl, hydroxy-alkyl, alkoxy-alkyl, cycloalkyl, cycloalkyl-alkyl, alkenyl, halo, haloalkyl, hydroxy, alkoxy, haloalkoxy, cyano, nitro and amino.

In a still more preferred embodiment Y represents cycloalkyl or phenyl, which phenyl is optionally substituted with alkyl, cycloalkyl, halo, trifluoromethyl, trifluoromethoxy, cyano, nitro or amino.

In a yet more preferred embodiment Y represents phenyl, optionally substituted with alkyl, cycloalkyl, halo, trifluoromethyl, trifluoromethoxy, cyano, nitro or amino.

In a further more preferred embodiment Y represents cycloalkyl or phenyl, which phenyl is optionally substituted with alkyl, in particular methyl, or with halo, in particular chloro.

In a still further more preferred embodiment Y represents phenyl, optionally substituted with alkyl, in particular methyl, ethyl or propyl, or halo, in particular fluoro, chloro or bromo.

In a still further more preferred embodiment Y represents cycloalkyl, in particular cyclohexyl, or phenyl, which phenyl is optionally substituted with methyl, fluoro, chloro or bromo.

In a still further more preferred embodiment Y represents phenyl, optionally substituted with methyl, fluoro, chloro or bromo.

In a still further more preferred embodiment Y represents cycloalkyl, in particular cyclohexyl, or phenyl, which phenyl is optionally substituted with methyl or chloro.

In a still further more preferred embodiment Y represents phenyl, optionally substituted with methyl or chloro.

In a still further more preferred embodiment Y represents cycloalkyl, in particular cyclopenyl, cyclohexyl or cycloheptyl.

In a still further more preferred embodiment Y represents cyclohexyl.

In a preferred embodiment the pyridinyl-pyrimidine derivative of the invention is a compound of Formula I, wherein R¹, R², R³, R⁴, R⁵ and R⁶, independently of each other, represent hydrogen, alkyl, cycloalkyl, cycloalkyl-alkyl, alkenyl, halo, haloalkyl, hydroxy, alkoxy, haloalkoxy, alkoxy-carbonyl, cyano, nitro, amino, phenyl or benzoyl.

In a more preferred embodiment R¹ and R⁵, independently of each other, represent hydrogen, alkyl, cycloalkyl, cycloalkyl-alkyl, alkenyl, halo, haloalkyl, hydroxy, alkoxy, haloalkoxy, alkoxy-carbonyl, cyano, nitro, amino, phenyl or benzoyl; and the remaining of R¹, R², R³, R⁴, R⁵ and R⁶ all represent hydrogen.

In an even more preferred embodiment R¹ and R⁵, independently of each other, represent hydrogen, alkyl, in particular methyl, ethyl or propyl, or trifluoromethyl; and the remaining of R¹, R², R³, R⁴, R⁵ and R⁶ all represent hydrogen.

In a still more preferred embodiment R¹ and R⁵, independently of each other, represent hydrogen, methyl or trifluoromethyl; and the remaining of R¹, R², R³, R⁴, R⁵ and R⁶ all represent hydrogen.

In a most preferred embodiment the pyridinyl-pyrimidine derivative of the invention is
(4-Chloro-phenyl)[2-(6-chloro-pyridin-2-yl)-6-methyl-pyrimidin-4-yl]-amine;
(4-Chloro-phenyl)-(6-methyl-2-pyridin-2-yl-pyrimidin-4-yl)-amine;
(4-Chloro-phenyl)-[2-(6-chloro-pyridin-2-yl)-pyrimidin-4-yl]-amine; or
Cyclohexyl-[6-methyl-2-(6-methyl-pyridin-2-yl)-pyrimidin-4-yl]-amine;

or a pharmaceutically acceptable salt thereof.

Any combination of two or more of the embodiments described herein is considered within the scope of the present invention.

### DEFINITION OF SUBSTITUENTS

In the context of this invention halo represents fluoro, chloro, bromo or iodo. Thus a trihalomethyl group represents e.g. a trifluoromethyl group, a trichloromethyl group, and similar trihalo-substituted methyl groups.

In the context of this invention a haloalkyl group designates an alkyl group as defined herein, which alkyl group is substituted one or more times with halo. Preferred haloalkyl groups of the invention include trihalomethyl, preferably trifluoromethyl.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl, and tertiary butyl. In a preferred embodiment of this invention alkyl represents a C₁₋₃-alkyl group, which may in particular be methyl, ethyl, propyl or isopropyl.

In the context of this invention an alkenyl group designates a carbon chain containing one or more double bonds, including di-enes, tri-enes and poly-enes. In a preferred embodiment the alkenyl group of the invention comprises of from two to eight carbon atoms (C₂₋₈-alkenyl), more preferred of from two to six carbon atoms (C₂₋₆-alkenyl), including at least one double bond. In a most preferred embodiment the alkenyl group of the invention is ethenyl; 1- or 2-propenyl; 1-, 2- or 3-butenyl, or 1,3-butenyl; 1-, 2-, 3-, 4- or 5-hexenyl, or 1,3-hexenyl, or 1,3,5-hexenyl; 1-, 2-, 3-, 4-, 5-, 6-, or 7-octenyl, or 1,3-octenyl, or 1,3,5-octenyl, or 1,3,5,7-octenyl.

In the context of this invention a hydroxy-alkyl group designates an alkyl group as defined above, which hydroxy-alkyl group is substituted with one or more hydroxy groups. Examples of preferred hydroxy-alkyl groups of the invention include 2-hydroxy-ethyl, 3-hydroxy-propyl, 4-hydroxy-butyl, 5-hydroxy-pentyl and 6-hydroxy-hexyl.

In the context of this invention a cycloalkyl group designates a cyclic alkyl group, preferably containing of from three to ten carbon atoms (C₃₋₁₀-cycloalkyl), preferably of from three to eight carbon atoms (C₃₋₈-cycloalkyl), including cyclopropyl, cyclobutyl, cyclopentyl, cyclohexyl, cycloheptyl and cyclooctyl.

In the context of this invention a cycloalkyl-alkyl group designates a cycloalkyl group as defined above, which cycloalkyl group is substituted on an alkyl group as also defined above. Examples of preferred cycloalkyl-alkyl groups of the invention include cyclopropylmethyl and cyclopropylethyl.

In the context of this invention an alkoxy group designates an “alkyl-O—” group, wherein alkyl is as defined above.

In the context of this invention a haloalkoxy group designates an alkoxy group as defined herein, which alkoxy group is substituted one or more times with halo. Preferred haloalkoxy groups of the invention include trihalomethoxy, preferably trifluoromethoxy.

In the context of this invention an amino-alkyl group designates an alkyl group as defined above, which alkyl group is substituted with an amino group. Examples of preferred amino-alkyl groups of the invention include 2-amino-ethyl, 3-amino-propyl, 4-amino-butyl, 5-amino-pentyl and 6-amino-hexyl.

In the context of this invention an alkyl-amino group designates a secondary (N-alkyl)amino group, monosubstituted with an alkyl group as defined above.

In the context of this invention an alkyl-amino-alkyl group designates an alkyl group as defined above, which alkyl group is substituted with a secondary (N-alkyl)amino group as defined above.

In the context of this invention an alkoxy-alkyl group designates an “alkyl-O-alkyl-” group, wherein alkyl is as defined above. Examples of preferred alkoxy-alkyl groups of the invention include methoxy-methyl, methoxy-ethyl, ethoxy-methyl, and ethoxy-ethyl.

### Steric Isomers

The pyridinyl-pyrimidine derivatives of the present invention may exist in (+) and (−) forms as well as in racemic forms. The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or l-(tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic add or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Moreover, some of the chemical compounds of the invention being oximes, may thus exist in two forms, syn- and anti-form (Z- and E-form), depending on the arrangement of the substituents around the —C═N— double bond. A chemical compound of the present invention may thus be the syn- or the anti-form (Z- and E-form), or it may be a mixture hereof.

### Pharmaceutically Acceptable Salts

The pyridinyl-pyrimidine derivatives of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulfonate derived from benzensulfonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulfonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a pyridinyl-pyrimidine derivative of the invention and its pharmaceutically acceptable acid addition salt.

Metal salts of a chemical compound of the invention include alkali metal salts, such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

The pyridinyl-pyrimidine derivative of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

### Methods of Preparation

The pyridinyl-pyrimidine derivatives of the invention may be prepared by conventional methods of chemical synthesis, e.g. those described in the working examples. The starting materials for the processes described in the present application are known or may readily be prepared by conventional methods from commercially available chemicals.

The end products of the reactions described herein may be isolated by conventional techniques, e.g. by extraction, crystallisation, distillation, chromato-graphy, etc.

### Biological Activity

The pyridinyl-pyrimidine derivatives of the invention have been subjected to in vitro experiments and found particularly useful as potassium channel modulating agents. More particularly the compounds of the invention are capable of selectively modulating SK1, SK2 and/or SK3 channels.

Therefore, in another aspect, the invention relates to the use of pyridinyl-pyrimidine derivatives of the invention for the manufacture of medicaments, which medicament may be useful for the treatment or alleviation of a disease or a disorder associated with the activity of potassium channels, in particular SK channels, more particularly SK1, SK2 and/or SK3 channels.

In a preferred embodiment, the disease or a disorder associated with the activity of potassium channels is a respiratory disease, epilepsy, convulsions, seizures, absence seizures, vascular spasms, coronary artery spasms, renal disorders, polycystic kidney disease, bladder spasms, urinary incontinence, bladder outflow obstruction, erectile dysfunction, gastrointestinal dysfunction, secretory diarrhoea, ischaemia, cerebral ischaemia, ischaemic heart disease, angina pectoris, coronary heart disease, autism, ataxia, traumatic brain injury, Parkinson's disease, bipolar disorder, psychosis, schizophrenia, anxiety, depression, mania, mood disorders, dementia, memory and attention deficits, Alzheimer's disease, amyotrophic lateral sclerosis (ALS), dysmenorrhea, narcolepsy, Reynaud's disease, intermittent claudication, Sjorgren's syndrome, arrhythmia, hypertension, myotonic muscle dystrophia, spasticity, xerostomi, diabetes type II, hyperinsulinemia, premature labour, baldness, cancer, irritable bowel syndrome, immune suppression, migraine or pain.

In a more preferred embodiment the disease or a disorder associated with the activity of potassium channels is a respiratory disease, urinary incontinence, erectile dysfunction, anxiety, epilepsy, psychosis, schizophrenia, amyotrophic lateral sclerosis (ALS) or pain.

In another preferred embodiment the disease or a disorder associated with the activity of potassium channels is a respiratory disease, in particular asthma, cystic fibrosis, chronic obstructive pulmonary disease (COPD) or rhinorrhea.

In a third preferred embodiment the disease or a disorder associated with the activity of potassium channels is urinary incontinence.

In a fourth preferred embodiment the disease or a disorder associated with the activity of potassium channels is epilepsy, seizures, absence seizures or convulsions.

In a fifth preferred embodiment the disease or a disorder associated with the activity of potassium channels is a respiratory disease, in particular asthma, cystic fibrosis, chronic obstructive pulmonary disease (COPD) or rhinorrhea.

The compounds tested all showed a biological activity in the micromolar and sub-micromolar range, i.e. of from below 1 to above 100 μM. Preferred compounds of the invention show a biological activity determined as described herein in the in the sub-micromolar and micromolar range, i.e. of from below 0.1 to about 10 μM.

### Pharmaceutical Compositions

In yet another aspect the invention provides novel pharmaceutical compositions comprising a therapeutically effective amount of the pyridinyl-pyrimidine derivatives of the invention.

In a most preferred embodiment the pyridinyl-pyrimidine derivative for use in a pharmaceutical composition of the invention is
4-Methyl-2-pyridin-2-yl-6-p-tolylsulfanyl-pyrimidine;
Phenyl-(2-pyridin-2-yl-6-trifluoromethyl-pyrimidin-4-yl)-amine;
(4-Chloro-phenyl)-[6-methyl-2-(6-methyl-pyridin-2-yl)-pyrimidin-4-yl]-amine;
(4-Chloro-phenyl)-[2-(6-chloro-pyridin-2-yl)-6-methyl-pyrimidin-4-yl]-amine;
(4-Chloro-phenyl)-(6-methyl-2-pyridin-2-yl-pyrimidin-4-yl)-amine;
(4-Chloro-phenyl)-(2-pyridin-2-yl-pyrimidin-4-yl)-amine;
(4-Chloro-phenyl)-[2-(6-chloro-pyridin-2-yl)-pyrimidin-4-yl]-amine; or
Cyclohexyl-[6-methyl-2-(6-methyl-pyridin-2-yl)-pyrimidin-4-yl]-amine;

or a pharmaceutically acceptable salt thereof.

While a pyridinyl-pyrimidine derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers and/or diluents.

In a preferred embodiment, the invention provides pharmaceutical compositions comprising the pyridinyl-pyrimidine derivative of the invention, or a pharmaceutically acceptable salt or derivative thereof, together with one or more pharmaceutically acceptable carriers therefor and, optionally, other therapeutic and/or prophylactic ingredients. The carrier(s) must be “acceptable” in the sense of being compatible with the other ingredients of the formulation and not deleterious to the recipient thereof.

Pharmaceutical compositions of the invention may be those suitable for oral, rectal, bronchial, nasal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The pyridinyl-pyrimidine derivative of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The pyridinyl-pyrimidine derivative according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilisers, buffers, artificial and natural sweeteners, dispersants, thickeners, solubilizing agents, and the like

For topical administration to the epidermis the chemical compound according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucrose and acacia; and mouthwashes comprising the active ingredient in a suitable liquid carrier.

Solutions or suspensions are applied directly to the nasal cavity by conventional means, for example with a dropper, pipette or spray. The compositions may be provided in single or multi-dose form. In the latter case of a dropper or pipette, this may be achieved by the patient administering an appropriate, predetermined volume of the solution or suspension. In the case of a spray, this may be achieved for example by means of a metering atomising spray pump.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

A therapeutically effective dose refers to that amount of active ingredient which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/ED₅₀. Pharmaceutical compositions which exhibit large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depend on the nature and severity of the disease being treated and the route of administration, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the prevention, treatment or alleviation of a disease or a disorder or a condition of a living animal body, including a human, which disease, disorder or condition is responsive to modulation of potassium channels, in particular SK channels, and which method comprises comprising administering to such a living animal body, including a human, in need thereof a therapeutically-effective amount of a pyridinyl-pyrimidine derivative of the invention.

The preferred indications contemplated according to the invention are those stated above.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

A satisfactory result can, in certain instances, be obtained at a dosage as low as 0.005 mg/kg i.v. and 0.01 mg/kg p.o. The upper limit of the dosage range is about 10 mg/kg i.v. and 100 mg/kg p.o. Preferred ranges are from about 0.001 to about 1 mg/kg i.v. and from about 0.1 to about 10 mg/kg p.o.

### EXAMPLES

The invention is further illustrated with reference to the following examples, which are not intended to be in any way limiting to the scope of the invention as claimed.

### Preparatory Examples

### Example 1

### 6-Methyl-2-(6-chloro-pyridin-2-yl)-pyrimidin-4-ol (Intermediate compound)

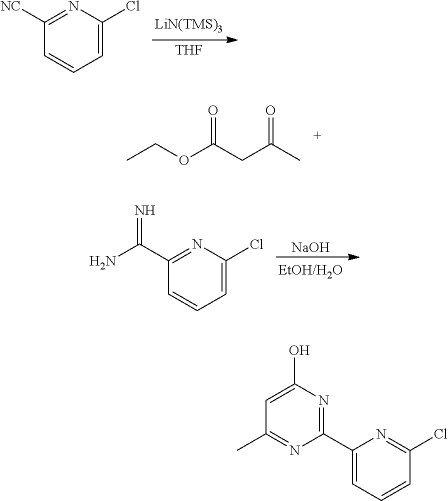

6-Chloro-2-pyridinecarbonitrile (500 mg, 3.61 mmol) was dissolved in tetra-hydrofuran (5 mL) and cooled to −78° C. Lithium bis(trimethylsilyl)amide (604 mg, 3.61 mmol) was added drop-wise. The reaction mixture was slowly warmed to room temperature and quenched with aqueous hydrogen chloride (1.5 N). The aqueous phase was washed with ethyl acetate and basified with 10% aqueous sodium bicarbonate.

Ethanol (20 mL) and ethyl acetoacetate (0.35 mL, 2.75 mmol) were added followed by addition of sodium hydroxide (200 mg, 5.01 mmol), and the mixture was stirred at room temperature for 3 days.

The reaction mixture was concentrated under reduced pressure to remove ethanol. The remaining aqueous layer was extracted with chloroform (3×25 mL). The combined organic phases were washed with water and brine, dried over anhydrous sodium sulphate filtrated and concentrated under reduced pressure to give 6-methyl-2-(6-chloro-pyridin-2-yl)-pyrimidin-4-ol (3 g, 35%) as an yellow solid.

### 2-(6-Chloro-pyridin-2-yl)-6-methyl-pyrimidin-4-ol (Intermediate compound)

Was prepared according to Example 1 from 6-methyl-2-pyridinecarbonitrile.

### Example 2

### 2-(6-Chloro-pyridin-2-yl)-pyrimidin-4-ol (Intermediate compound)

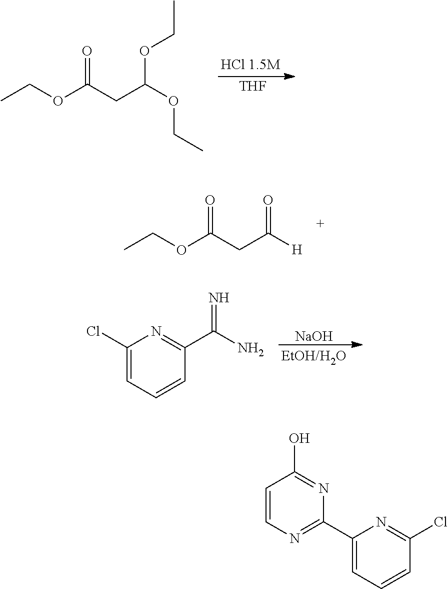

Ethyl-3,3-diethoxypropionate (1 g, 5.26 mL) was dissolved in tetrahydrofuran (2 mL) and aqueous hydrochloric acid (1.5 M) was added. The reaction mixture was stirred at room temperature for 4 hours and extracted with ethyl acetate. The combined organic layers were washed with brine, dried over sodium sulphate, filtrated and evaporated. Ethanol (20 mL) was added and cooled to 0° C. Crude 6-chloro-pyridine-2-carboxamidine (560 mg, 1.44 mmol) in water (5 mL) was added and the reaction mixture was stirred for 15 minutes at 0° C. Sodium hydroxide (841 mg, 21.03 mmol) was added followed by additional stirring at room temperature for 20 hours. The reaction mixture was concentrated under reduced pressure. Water was added and extracted with chloroform. The combined organic phases were washed with brine, dried over sodium sulphate filleted and evaporated to give 2-(6-chloro-pyridin-2-yl)-pyrimidin-4-ol (350 mg, 53%) as a brownish solid.

### Example 3

### 4-Chloro-6-methyl-2-(6-chloro-pyridin-2-yl)-pyrimidine (Intermediate compound)

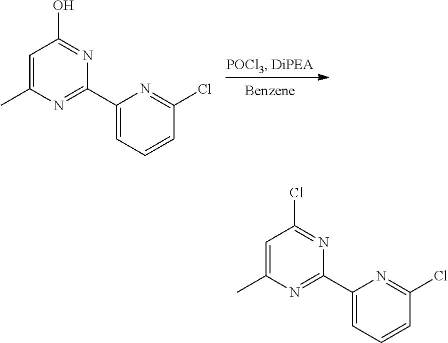

6-Methyl-2-(6-methyl-pyridin-2-yl)-pyrimidin-4-ol (150 mg, 0.68 mmol) was dissolved in benzene (3 mL) and N,N-diisopropylethylamine (0.18 mL, 1.02 mmol) was added and the temperature raised to 80° C. Phosphorous oxychloride (63.1 μL, 0.68 mmol) was carefully added and the reaction mixture was heated to 110° C. for 4 hours. The reaction mixture was then cooled to 0° C. and quenched with a small amount of ice, basified with sodium bicarbonate and filtrated. The residue was washed with chloroform and the filtrate was extracted with chloroform. The combined organic layers washed with water and brine, dried over anhydrous sodium sulphate and concentrated under reduced pressure to give 4-cloro-6-methyl-2-(6-chloro-pyridin-2-yl)-pyrimidine (120 mg, 75%) as a brown solid.

### 4-Chloro-2-(6-chloro-pyridin-2-yl)-pyrimidine (Intermediate compound)

Was prepared according to Example 3 from 2-(6-chloro-pyridin-2-yl)-pyrimidin-4-ol.

### 4-Chloro-2-(6-chloro-pyridin-2-yl)-6-methyl-pyrimidine (Intermediate compound)

Was prepared according to Example 3 from 2-(6-chloro-pyridin-2-yl)-6-methyl-pyrimidin-4-ol.

### Example 4

### (4-Chloro-phenyl)-[6-methyl-2-(6-chloro-pyridin-2-yl)-pyrimidin-4-yl]-amine (Compound 4A)

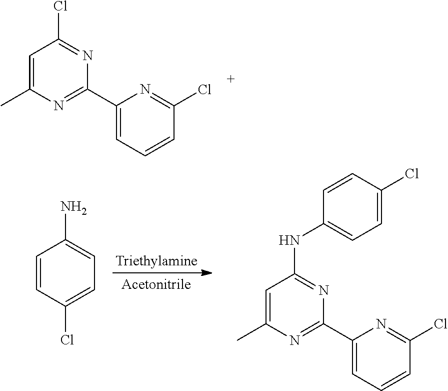

4-Chloro-6-methyl-2-(6-chloro-pyridin-2-yl)-pyrimidine (150 mg, 0.62 mmol), triethylamine (131 μL, 0.94 mmol), 4-chloroaniline (79.7 mg, 0.62 mmol) was dissolved in acetonitrile (4 mL) and heated in a microwave oven for 1 hour at 180° C. The reaction mixture was concentrated under reduced pressure. The crude product was purified by column chromatography using chloroform with 1.5% of methanol as eluent. The product contained triethylamine and was dissolved in chloroform and washed with water. The organic layer was dried with anhydrous sodium sulphate, fliterated and concentrated in vacuo. Hydrochloric acid in ether was added to give (4-chloro-phenyl)-[6-methyl-2-(6-chloro-pyridin-2-yl)-pyrimidin-4-yl]-amine (19.7 mg, 9%) as the hydro-chloride. Mp=239° C. LC-ESI-HRMS of [M+H]+ shows 331.0503 Da. Calc. 331.051727 Da, dev. −4.3 ppm.

### (4-Chloro-phenyl)-[2-(6-chloro-pyridin-2-yl)-pyrimidin-4-yl]-amine (Compound 4B)

Was prepared according to Example 4 from 4-chloro-2-(6-chloro-pyridin-2-yl)-pyrimidine and 4-chloroaniline. Mp. 174-176° C.

### (4-Chloro-phenyl)-(6-methyl-2-pyridin-2-yl-pyrimidin-4-yl)-amine (Compound 4C)

Was prepared according to Example 4 from 4-chloro-2-(6-chloro-pyridin-2-yl)-6-methyl-pyrimidine and 4-chloroaniline. ¹H NMR (CDCl₃) δ 2.49 (s, 3H), 6.59 (s, 1H), 7.21 (br s, 1H), 7.30-7.39 (m, 5H), 7.83 (t, J=7.77 Hz, 1H), 8.43 (d, J=7.91 Hz, 1H), 8.80 (d, J=4.19, 1H).

### Example 5

### Cyclohexyl-[6-methyl-2-(6-methyl-pyridin-2-yl)-pyrimidin-4-yl]-amine (Compound 5)

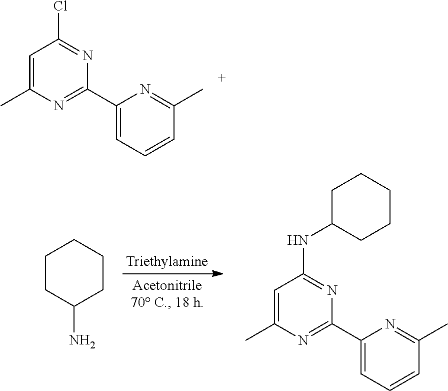

4-Chloro-2-(6-chloro-pyridin-2-yl)-6-methyl-pyrimidine (500 mg, 2.28 mmol), cyclohexylamine (0.26 mL, 2.28 mmol) and triethylamine (0.48 mL, 3.41 mmol) were dissolved in acetonitrile. The reaction mixture was heated on a sand bath at 70° C. for 18 hours and then concentrated in vacuo. The crude product was purified by column chromatography using chloroform containing 1% methanol as eluent to give cyclohexyl-[6-methyl-2-(6-methyl-pyridin-2-yl)-pyrimidin-4-yl]-amine (80.8 mg, 13%). Mp. 59.6-65° C.

### Example 6

### Biological Activity

This example demonstrates the biological activity of a compound representative of the invention (Compound 4A). The ionic current through small-conductance Ca²⁺-activated K⁺ channels (SK channels, subtype 3) is recorded using the whole-cell configuration of the patch-clamp technique.

HEK293 tissue culture cells expressing hSK3 channels were grown in DMEM (Dulbecco's Modified Eagle Medium) supplemented with 10% FCS (foetal calf serum) at 37° C. in 5% CO₂. At 60-80% confluency, cells were harvested by trypsin treatment and seeded on cover slips.

Experiments are carried out on one of several patch-clamp set-ups. Cells plated on coverslips are placed in a 15 μl perfusion chamber (flowrate ˜1 ml/min) mounted on an IMT-2 microscope. The microscopes are placed on vibration-free tables in grounded Faraday cages. All experiments are performed at room temperature (20-22° C.). EPC-9 patch-clamp amplifiers (HEKA-electronics, Lambrect, Germany) are connected to Macintosh computers via ITC16 interfaces. Data are stored directly on the hard-disk and analysed by IGOR software (Wavemetrics, Lake Oswega, Oreg., USA).

The whole-cell configuration of the patch-clamp technique is applied. In short: The tip of a borosilicate pipette (resistance 2-4 MΩ) is gently placed on the cell membrane using remote control systems. Light suction results in the formation of a giga seal (pipette resistance increases to more than 1 GΩ) and the cell membrane underneath the pipette is then ruptured by more powerful suction. Cell capacitance is electronically compensated and the resistance between the pipette and the cell interior (the series resistance, Rs) is measured and compensated for. Usually the cell capacitance ranges from 5 to 20 pF (depending on cell size) and the series resistance is in the range 3 to 6 MΩ. Rs- as well as capacitance compensation are updated during the experiments (before each stimulus). All experiments with drifting Rs-values are discharged. Leak-subtractions are not performed.

The extracellular (bath) solution contains (in mM): 154 mM KCl, 0.1 CaCl₂, 3 MgCl₂, 10 HEPES (pH=7.4 with HCl). The test compound was dissolved 1000 times in DMSO from a concentrated stock solution and then diluted in the extracellular solution.

The intracellular (pipette) solution contained: 154 mM KCl, 10 mM HEPES, 10 mM EGTA. Concentrations of CaCl₂ and MgCl₂ needed to obtain the desired free concentrations of Ca²⁺ (0.3-0.4 μM, Mg²⁺ always 1 mM) were calculated by EqCal software (Cambridge, UK) and added.

After establishment of the whole-cell configuration, voltage-ramps (normally −80 to +80 mV) are applied to the cell every 5 seconds from a holding potential of 0 mV. A stable baseline current is obtained within a period of 100-500 seconds, and the compound is then added by changing to an extracellular solution containing the test compound. Active compounds are quantified by calculating the change in baseline current at −75 mV. The current in the absence of compound is set to 100%. Activators will have values greater than 100, and a value of 200% indicates a doubling of the current. On the other hand, a value of 50% indicates that the compound has reduced the baseline current to half its value.

For activators a SC₁₀₀ value may be estimated. The SC₁₀₀ value is defined as the Stimulating Concentration required for increasing the baseline current by 100%. The SC₁₀₀ value determined for Compound 4A of the invention was 0.3 μM, which is an indication of its strong SK3 activating properties.

1. A pyridinyl-pyrimidine derivative of Formula I 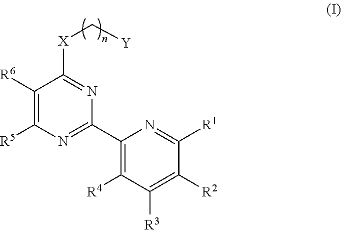
an enantiomer or a mixture of its enantiomers, an N-oxide thereof, a prodrug thereof, or a pharmaceutically acceptable salt thereof, wherein
n is 0, 1, 2 or 3;
X represents O, S or NR′; wherein
R′ represents hydrogen, alkyl, cycloalkyl or cycloalkyl-alkyl;
Y represents alkyl, cycloalkyl, alkyl-cycloalkyl, alkenyl or phenyl, which phenyl is optionally substituted one or more times with substituents selected from the group consisting of alkyl, amino-alkyl, alkyl-amino, alkyl-amino-alkyl, hydroxy-alkyl, alkoxy-alkyl, cycloalkyl, cycloalkyl-alkyl, alkenyl, halo, haloalkyl, hydroxy, alkoxy, haloalkoxy, cyano, nitro and amino; and
R¹, R², R³, R⁴, R⁵ and R⁶, independently of each other, represent hydrogen, alkyl, cycloalkyl, cycloalkyl alkyl, alkenyl, halo, haloalkyl, hydroxy, alkoxy, haloalkoxy, alkoxy-carbonyl, cyano, nitro, amino, phenyl or benzoyl.

2. The pyridinyl-pyrimidine derivative of claim 1, an enantiomer or a mixture of its enantiomers, an N-oxide thereof, a prodrug thereof, or a pharmaceutically acceptable salt thereof, wherein n is 0, 1, 2 or 3.

3. The pyridinyl-pyrimidine derivative of claim 1, an enantiomer or a mixture of its enantiomers, an N-oxide thereof, a prodrug thereof, or a pharmaceutically acceptable salt thereof, wherein
X represents O, S or NR′; wherein
R′ represents hydrogen, alkyl, cycloalkyl or cycloalkyl-alkyl.

4. The pyridinyl-pyrimidine derivative of claim 1, an enantiomer or a mixture of its enantiomers, an N-oxide thereof, a prodrug thereof, or a pharmaceutically acceptable salt thereof, wherein
Y represents alkyl, cyclo alkyl, alkyl-cycloalkyl, alkenyl or phenyl, which phenyl is optionally substituted one or more times with substituents selected from the group consisting of alkyl, amino-alkyl, alkyl-amino, alkyl-amino-alkyl, hydroxy-alkyl, alkoxy-alkyl, cycloalkyl, cycloalkyl-alkyl, alkenyl, halo, haloalkyl, hydroxy, alkoxy, haloalkoxy, cyano, nitro and amino.

5. The pyridinyl-pyrimidine derivative of claim 1, an enantiomer or a mixture of its enantiomers, an N-oxide thereof, a prodrug thereof, or a pharmaceutically acceptable salt thereof, wherein
R¹, R², R³, R⁴, R⁵ and R⁶, independently of each other, represent hydrogen, alkyl, cycloalkyl, alkenyl, halo, haloalkyl, hydroxy, alkoxy, haloalkoxy, alkoxy-carbonyl, cyano, nitro, amino, phenyl or benzoyl.

6. The pyridinyl-pyrimidine derivative of claim 1, which is
(4-Chloro-phenyl)-[2-(6-chloro-pyridin-2-yl)-6-methyl-pyrimidin-4-yl]-amine;
(4-Chloro-phenyl)-(6-methyl-2-pyridin-2-yl-pyrimidin-4-yl)-amine;
(4-Chloro-phenyl)-[2-(6-chloro-pyridin-2-yl)-pyrimidin-4-yl]-amine; or
Cyclohexyl-[6-methyl-2-(6-methyl-pyridin-2-yl)-pyrimidin-4-yl]-amine;
or a pharmaceutically acceptable salt thereof.

7. A pharmaceutical composition comprising a therapeutically-effective amount of a pyridinyl-pyrimidine derivative according to claim 1, an enantiomer or a mixture of its enantiomers, an N-oxide thereof, or a pharmaceutically-acceptable salt thereof, or a prodrug thereof, together with at least one pharmaceutically-acceptable carrier or diluent.

8. The pharmaceutical composition of claim 7, wherein the pyridinyl-pyrimidine derivative is
4-Methyl-2-pyridin-2-yl-6-p-tolylsulfanyl-pyrimidine;
Phenyl-(2-pyridin-2-yl-6-trifluoromethyl-pyrimidin-4-yl)-amine;
(4-Chloro-phenyl)-[6-methyl-2-(6-methyl-pyridin-2-yl)-pyrimidin-4-yl]-amine;
(4-Chloro-phenyl)-[2-(6-chloro-pyridin-2-yl)-6-methyl-pyrimidin-4-yl]-amine;
(4-Chloro-phenyl)-(6-methyl-2-pyridin-2-yl-pyrimidin-4-yl)-amine;
(4-Chloro-phenyl)-(2-pyridin-2-yl-pyrimidin-4-yl)-amine;
(4-Chloro-phenyl)-[2-(6-chloro-pyridin-2-yl)-pyrimidin-4-yl]-amine; or
Cyclohexyl-[6-methyl-2-(6-methyl-pyridin-2-yl)-pyrimidin-4-yl]-amine;
or a pharmaceutically acceptable salt thereof

9. A method of treatment, prevention or alleviation of a disease or a disorder or a condition of a living animal body, including a human, which disease, disorder or condition is responsive to modulation of the potassium channels, and which method comprises con rip administering to such a living animal body, including a human, in need thereof a therapeutically-effective amount of a pyridinyl-pyrimidine derivative of claim 1.

10. The method according to claim 9, wherein the disease or a disorder associated with the activity of potassium channels is a respiratory disease, epilepsy, convulsions, seizures, absence seizures, vascular spasms, coronary artery spasms, renal disorders, polycystic kidney disease, bladder spasms, urinary incontinence, bladder outflow obstruction, erectile dysfunction, gastrointestinal dysfunction, secretory diarrhoea, ischaemia, cerebral ischaemia, ischaemic heart disease, angina pectoris, coronary heart disease, autism, ataxia, traumatic brain injury, Parkinson's disease, bipolar disorder, psychosis, schizophrenia, anxiety, depression, mania, mood disorders, dementia, memory and attention deficits, Alzheimer's disease, amyotrophic lateral sclerosis (ALS), dysmenoirhea, narcolepsy, Reynaud's disease, intermittent claudication, Sjorgren's syndrome, arrhythmia, hypertension, myotonic muscle dystrophia, spasticity, xerostomi, diabetes type II, hyperinsulinemia, premature labour, baldness, cancer, irritable bowel syndrome, immune suppression, migraine or pain.

11. The method according to claim 9, wherein the disease or a disorder associated with the activity of potassium channels is a respiratory disease, urinary incontinence, erectile dysfunction, anxiety, epilepsy, psychosis, schizophrenia, amyotrophic lateral sclerosis (ALS) or pain.

12. The methodaccording to claim 9, wherein the activity of potassium channels is a respiratory disease, in particular asthma, cystic fibrosis, chronic obstructive pulmonary disease (COPD) or rhinorrhea.

13. (canceled)
